(19) United States
(12) Patent Application Publication
(10) Pub. No.: US 2010/0056916 A1
(43) Pub. Date: Mar. 4, 2010

(54) SYSTEM, DEVICE, METHOD, COMPUTER-READABLE MEDIUM, AND USE FOR IN VIVO IMAGING OF TISSUE IN AN ANATOMICAL STRUCTURE

(75) Inventors: Levinus Peter Bakker, Eindhoven (NL); Michael Cornelis Van Beek, Eindhoven (NL); Martinus Bernardus Van Der Mark, Eindhoven (NL); Rene Van Den Ham, Eindhoven (NL); Bernardus Hendrikus Wilhelmus Hendriks, Eindhoven (NL); Ralf Hoffmann, Eindhoven (NL); Nijs Cornelis Van Der Vaart, Eindhoven (NL); Marjolein Van Der Voort, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/515,023

(22) PCT Filed: Nov. 16, 2007
(86) PCT No.: PCT/IB07/54674

§ 371 Date: May 15, 2009

(30) Foreign Application Priority Data
Nov. 21, 2006 (EP) 06124440.6

(51) Int. Cl. A61B 8/14; A61B 6/00

(52) U.S. Cl. 600/443; 600/476

(57) ABSTRACT

A system is provided that may be used for locating and diagnosing lesions in the human body in vivo. In some embodiments, once the exact position of a lesion is found, a biopsy may be taken from the lesion using e.g. ultra sound techniques for guidance of the biopsy needle. Use of the system drastically reduces the negative biopsy samples compared to currently used “blind sampling” techniques. This reduces patient discomfort and minimizes infections as the number of biopsy samples is reduced. A method and computer-readable medium is also provided.

### FIELD OF THE INVENTION

This invention pertains in general to the field of medical imaging. More particularly the invention relates to imaging of different tissue types in vivo and guiding a tissue biopsy using medical imaging.

### BACKGROUND OF THE INVENTION

Prostate cancer is the most common cancer in men excluding skin cancer. The American Cancer Society, ACS, estimates that about 232,090 new cases of prostate cancer will be diagnosed in the United States and 30,350 men will die of this disease in 2005. The ACS estimates that a male in the US has a 1 in 6 risk of developing prostate cancer during his lifetime.

There are several tests are available for detection of prostate cancer, such as, Prostate-specific antigen (PSA) blood test, Digital Rectal Exam (DRE), Transrectal Ultrasound (TRUS) and Core Needle Biopsy. PSA, DRE and TRUS have limited sensitivity and/or specificity to the lesions and are mainly used to estimate the risk of having prostate cancer, depending on size and shape etc. The diagnosis of prostate cancer is usually performed using a biopsy in which a small sample of prostate tissue is removed and examined under a microscope. The main method for taking a prostate biopsy is a Core Needle Biopsy using TRUS for guidance. The biopsy is required to diagnose and determine the stage of prostate cancer. If a biopsy is taken from a tumor, the pathologist may diagnose cancer with a very high accuracy. However, a problem is to take a biopsy from the correct tissue volume. At the moment TRUS is used as an imaging modality to image diseased tissue. The TRUS systems may also be used to guide a biopsy from the diseased tissue volume. In some cases it is possible to recognize lesions using TRUS, however in many cases no lesions are visible, and in these cases TRUS may only be used to determine the position and size of the prostate. Since the position of the lesion is not known, multiple biopsies, typically between 6 and 13, are taken randomly, in an attempt to encounter at least one of the present tumor lesions. Obviously, this procedure leads to numerous false negatives.

EP 1 559 363 A2 discloses a system combining optical imaging technologies with anatomical imaging technologies (e.g. MR, ultrasound). The system can be used for image guidance that may include guiding a biopsy. A drawback of the system is that the optical imaging technology presented, i.e. fluorescence imaging, therein only has a rather limited penetration depth. Hence, lesions located deep from the surface of an investigated tissue may not be detected using EP 1 559 363 A2.

Hence an improved system, method, computer-readable medium, and use would be advantageous.

### SUMMARY OF THE INVENTION

Accordingly, the present invention preferably seeks to mitigate, alleviate or eliminate one or more of the above-identified deficiencies in the art and disadvantages singly or in any combination and solves at least the above-mentioned problems by providing a system, a method, and a computer-readable medium according to the appended patent claims.

According to one aspect of the invention, a system for in vivo imaging of tissue in an anatomical structure is provided. The system comprises a first unit connected to at least one electromagnetic radiation source for emitting pulsed electromagnetic radiation into the anatomical structure, whereby a first ultrasonic acoustic wave is generated from the tissue, the system further comprising at least one ultrasound source for emitting a second ultrasonic acoustic wave into the anatomical structure, at least one detector unit for receiving the first ultrasonic acoustic wave and the second ultrasonic acoustic wave, an image reconstruction unit for reconstructing a first image dataset of the tissue based on the received first ultrasonic acoustic wave and a second image dataset of the tissue based on the received second ultrasonic acoustic wave.

According to another aspect of the invention, a method for imaging of tissue in an anatomical structure is provided. The method comprises emitting electromagnetic radiation into the anatomical structure from at least one electromagnetic radiation source, generating a first ultrasonic acoustic wave from the tissue, receiving the first ultrasonic acoustic wave, reconstructing a first image dataset of the tissue based on the received first ultrasonic acoustic wave, emitting a second ultrasonic acoustic wave into the anatomical structure, receiving the second ultrasonic acoustic wave, and reconstructing a second image dataset of the tissue based on the received second ultrasonic acoustic wave.

According to a further aspect of the invention, a computer-readable medium having embodied thereon a computer program for processing by a computer for imaging of tissue in an anatomical structure is provided. The computer program comprises a first emission code segment for emitting electromagnetic radiation into the anatomical structure from at least one electromagnetic radiation source, whereby a first ultrasonic acoustic wave is generated from the tissue, a first reception code segment for receiving the first ultrasonic acoustic wave, a first reconstruction code segment for reconstructing a first image dataset of the tissue based on the received first ultrasonic acoustic wave a second emission code segment for emitting a second ultrasonic acoustic wave into the anatomical structure, a second reception code segment for receiving the second ultrasonic acoustic wave, a second reconstruction code segment for reconstructing a second image dataset of the tissue based on the received second ultrasonic acoustic wave.

According to yet another aspect of the invention, a use of the system according to any of the claims 1-13 is provided for locating and diagnosing a lesion in a tissue in an anatomical structure in vivo.

According to another aspect of the invention, a use of the system according to any of the claims 1-13 is provided for guiding a biopsy of a lesion in a tissue in an anatomical structure in vivo.

Embodiments of the present invention pertain to the use of Photoacoustic Imaging for creating an image dataset for the detection of suspicious prostate tissue. The system according to some embodiments may be used to guide a biopsy thereby reducing the number of false negatives, as the location of diseased tissue becomes known.

In some embodiments the present invention utilizes photoacoustic functionality in a transrectal unit in order to differentiate between lesions and healthy tissue. This means adding means to illuminate the prostate tissue with pulsed electromagnetic radiation, for instance using an optical fiber and a pulsed laser.

### BRIEF DESCRIPTION OF THE DRAWINGS

These and other aspects, features and advantages of which the invention is capable of will be apparent and elucidated from the following description of embodiments of the present invention, reference being made to the accompanying drawings, in which

FIG. 1 is a block diagram of a system according to an embodiment;

FIG. 2 is a diagram showing the difference of absorption spectrum for healthy and cancerous tissue;

FIG. 3 is a cross-sectional view of a system according to an embodiment;

FIG. 4 is a cross-sectional view of a system according to another embodiment;

FIG. 5 is a block diagram of a method according to an embodiment; and

FIG. 6 is a block diagram of a computer-readable medium according to an embodiment.

### DETAILED DESCRIPTION OF THE EMBODIMENTS

Several embodiments of the present invention will be described in more detail below with reference to the accompanying drawings in order for those skilled in the art to be able to carry out the invention. The invention may, however, be embodied in many different forms and should not be construed as limited to the embodiments set forth herein. Rather, these embodiments are provided so that this disclosure will be thorough and complete, and will fully convey the scope of the invention to those skilled in the art. The embodiments do not limit the invention, but the invention is only limited by the appended patent claims. Furthermore, the terminology used in the detailed description of the particular embodiments illustrated in the accompanying drawings is not intended to be limiting of the invention.

The following description focuses on embodiments of the present invention applicable to an imaging system and in particular to an imaging system for imaging of diseased tissue in vivo and for guiding a tissue biopsy.

The present invention utilizes Photoacoustic Imaging to image tissue in vivo, such as the prostate. Using Photoacoustic Imaging the optical properties of tissue in the near infrared region may be determined. Photoacoustic imaging is sensitive to absorption by for instance water, lipid, hemoglobin (Hb) and oxyhemoglobin (HbO2). Diseased tissue differs from normal tissue in the concentration of these substances. As diseased tissue, such as malignant tissue, might comprise higher relative water content, than normal tissue the present invention is according to some embodiments able to distinguish between healthy and diseased tissue.

The present invention provides embodiments for creating an image dataset illustrating the water, lipid, Hb, and HbO2 content of tissue in vivo. As the optical properties of tissue are different for malignant and healthy tissue, the created image dataset will contain information that may be used to distinguish between malignant and healthy tissue.

Photoacoustic Imaging is a non-invasive medical imaging technique, based upon the photoacoustic effect, which may be used for visualizing the internal structure and function of soft tissues, such as the prostate, and other possible applications include imaging the breast for the diagnosis and screening of cancer, the assessment of vascular disease and imaging skin abnormalities such as melanoma and vascular lesions. The technique relies upon irradiating the soft tissue of interest with preferably nanosecond pulses of low energy laser light. At near infrared wavelengths, due to the relative optical transparency of tissue, the electromagnetic radiation penetrates deeply, such as several cm. It is also strongly scattered. This results in a relatively large volume of the tissue becoming “bathed” in diffuse electromagnetic radiation. Through the processes of optical absorption and thermo elastic expansion, i.e. heating of the tissue, broadband (˜30 MHz) ultrasonic acoustic waves are excited or generated throughout the irradiated volume and propagate outwards. Here, as in conventional pulse-echo ultrasound, they can be detected using an array of ultrasound detectors or acoustic receivers and be spatially resolved to provide a 3D image of the internal tissue structure.

An advantage of Photoacoustic Imaging over other imaging modalities is based upon the strong optical contrast of different tissue types offering the prospect of identifying anatomical features that are indistinguishable using other radiological modalities such as ultrasound imaging or X-ray imaging. Compared to other common imaging modalities, such as MRI, Photoacoustic Imaging is a much cheaper imaging modality. As an example hemoglobin and its various states provides strong optical contrast at NIR and visible wavelengths making the technique well suited to imaging blood vessels—by comparison, the contrast of conventional ultrasound images tends to be limited by the relatively poor echogenicity, i.e. the ability to create an echo meaning returning a signal in ultrasound examinations, of blood vessels. In addition to the direct visualization of blood vessels the high contrast offered by hemoglobin provides the opportunity to indirectly detect abnormalities such as cancerous lesions that are accompanied by characteristic changes in the surrounding vasculature through angiogenesis. Additional advantages of the technique are that as a non-ionizing technique it avoids the safety concerns associated with X-ray imaging and has the potential to be configured as a relatively inexpensive portable instrument for bedside use or screening purposes.

The system, method, and computer-readable medium according to some embodiments of the invention provides at least one of enhanced imaging resolution, increased detection of diseased tissue, imaging penetration depth, flexibility, cost effectiveness, and less strain to affected subjects.

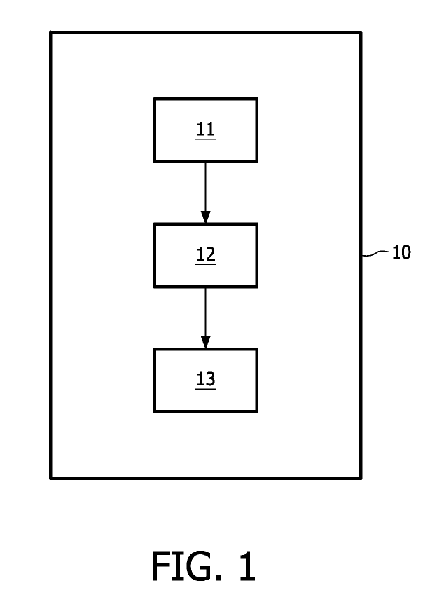

In an embodiment, according to FIG. 1, a system 10 for imaging of tissue in an anatomical structure in vivo is provided. The system comprises at least one electromagnetic radiation source 11 for emitting incident electromagnetic radiation on the anatomical structure. As the electromagnetic radiation propagates through the anatomical structure it is absorbed in the tissue due to the optical characteristics in the tissue. This results in thermo elastic expansion of the tissue, which will result in that broadband ultrasonic acoustic waves are excited throughout the irradiated tissue and will propagate outwards from the tissue. Different tissue has different optical characteristics and hence the electromagnetic radiation scatters and is absorbed differently depending on the tissue type. The system further comprises at least one detector unit 12 for receiving the ultrasonic acoustic waves. Furthermore, the system comprises an image reconstruction unit 13 for reconstructing an image dataset of the tissue based on the received ultrasonic acoustic waves by detector. The resulting image dataset will contain information of the water, lipid and (oxy)-hemoglobin content of the tissue at different locations of the tissue, and as different types of tissue contain different concentrations of these substances, tissue type and the location of the tissue type may be calculated from the image dataset. Accordingly, the system may be used to distinguish between different tissue types in vivo. The tissue type may be characterized as healthy and diseased tissue, such as healthy prostate cells and malignant prostate cells, respectively. Hence an advantage of this embodiment is that diseased tissue, such as lesions, may be accurately detected. Furthermore, this embodiment provides a way of detecting suspicious tissue located more than 1 mm beneath the surface of the tissue may be detected.

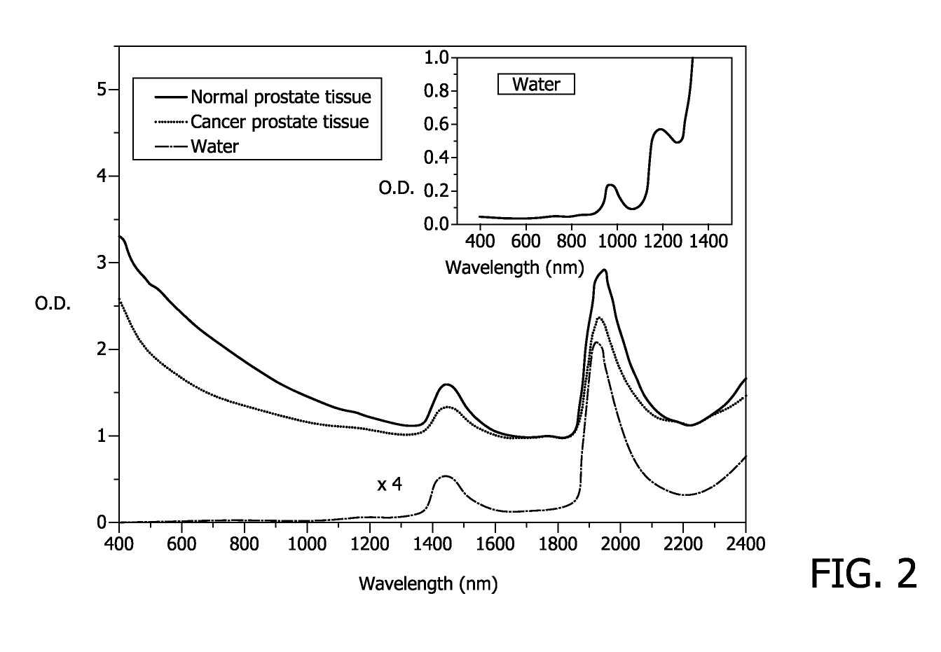

FIG. 2 [by R. R Alfano, et al, US 2005/0240107A1] shows the difference in absorption spectrum between normal, i.e. healthy, and cancerous prostate tissue. The differences are clearly visible. By using either a single or a multiple of wavelengths in the regions where there is a clear difference between healthy and cancerous tissue (for instance at 400-1000 nm), the tumor can be located.

### Image Reconstruction

In an embodiment the image reconstruction unit utilizes an imaging technique comprising for instance back projection, in which the time-dependent photoacoustic signals detected are spatially resolved by use of the speed of sound and back projected over hemispherical surfaces to obtain a 3D image of the initial pressure distribution.

In an embodiment the image reconstruction utilizes an image reconstruction algorithm for obtaining a resulting 3D image dataset of the tissue.

The image reconstruction unit may be any unit normally used for performing the involved tasks, e.g. a hardware, such as a processor with a memory. The processor may be any of variety of processors, such as Intel or AMD processors, CPUs, microprocessors, Programmable Intelligent Computer (PIC) microcontrollers, Digital Signal Processors (DSP), etc. However, the scope of the invention is not limited to these specific processors. The memory may be any memory capable of storing information, such as Random Access Memories (RAM) such as, Double Density RAM (DDR, DDR2), Single Density RAM (SDRAM), Static RAM (SRAM), Dynamic RAM (DRAM), Video RAM (VRAM), etc. The memory may also be a FLASH memory such as a USB, Compact Flash, SmartMedia, MMC memory, MemoryStick, SD Card, MiniSD, MicroSD, xD Card, TransFlash, and MicroDrive memory etc. However, the scope of the invention is not limited to these specific memories.

In an embodiment the system is comprised in a medical workstation or medical system, such as a Computed Tomography (CT) system, Magnetic Resonance Imaging (MRI) System or Ultrasound Imaging (US) system.

### Detector Unit

In an embodiment the detector unit is an ultrasound detector, comprising at least one piezoelectric element, for converting the detected ultrasonic acoustic wave into an electric signal. Other examples of detector units include, but are not limited to, Capacitive Micro-machined Ultrasonic Transducers (cMUT) technology and Piezoelectric Micro-machined Ultrasonic Transducers (pMUT).

In another embodiment the detector unit comprises one or more detector arrays, such as rectangular arrays comprising several elements. Another embodiment is a 1 dimensional array with mechanical scanning in the perpendicular direction.

In a further embodiment the detector unit comprises a combination of optical detectors and ultrasonic detectors. The optical detectors, such as monochrome and color Charged Coupled Device CCD chips or Complimentary Metal-Oxide Semiconductor CMOS chips, may be used to analyze electromagnetic radiation that has been scattered in the tissue and then propagated towards the optical detectors. Monochrome optical detectors have no intrinsic capability of analyzing individual wavelengths of the received electromagnetic radiation. If spectral analysis of the electromagnetic radiation is desired additional optics components such as lenses, gratings or prisms may be used to provide refraction of the received electromagnetic radiation before hitting the detector chip in order to be able to identify the wavelength spectrum of the received electromagnetic radiation and hence provide this information to the image reconstruction unit.

In another embodiment the electromagnetic radiation source emits electromagnetic radiation at several wavelengths sequentially, and the detector unit detects the incident electromagnetic radiation separately for each utilized wavelength.

### Electromagnetic Radiation Source

In an embodiment the electromagnetic radiation source emits electromagnetic radiation of a single wavelength, i.e. the electromagnetic radiation source having a narrow wavelength spectrum, such as solid state lasers such as Nd:YAG lasers (1064 nm), semiconductor lasers such as commercial laser diodes (375 nm-1800 nm) and Ti:Saphire lasers.

In an embodiment the electromagnetic radiation source is a solid-state laser.

In an embodiment the electromagnetic radiation source is a semiconductor laser.

In an embodiment the electromagnetic radiation source emits electromagnetic radiation comprising wavelengths in the near infrared region.

In other embodiments the electromagnetic radiation source may be pulsed light-emitting diodes.

In an embodiment alternatively the electromagnetic radiation sources emit electromagnetic radiation that excites the electrons in the atoms of the tissue to a higher energy state. When the electrons returns to a lower energy state the excess energy will be in the form of fluorescence light. Hence, if the detector unit comprises an optical detector suitable for receiving fluorescence light it may be used in fluorescence mode. In this case filters are used to block the excitation light. The detected fluorescence light may be auto-fluorescence from the tissue or fluorescence from an exogenous contrast agent. The detected fluorescence signal depends on the concentration and distribution of the fluorophores and on the scattering and absorption properties of the tissue. An exogenous contrast agent may be advantageous to use in photoacoustic imaging when it significantly changes the absorption of the lesion with respect to the healthy tissue.

### Probe Unit

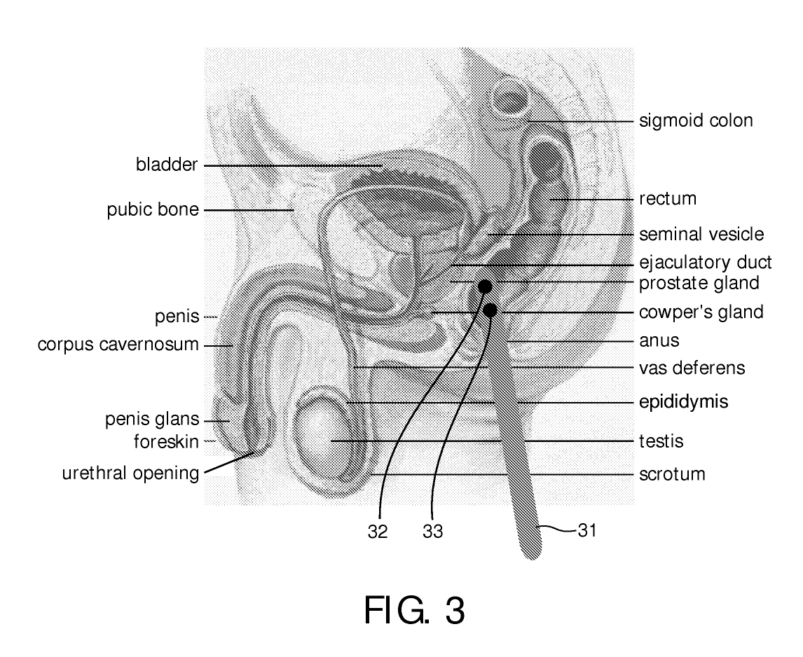

In an embodiment, according to FIG. 3, the system further comprises a probe unit 31, in which all of the electromagnetic radiation sources 32 and detectors 33 of the system are comprised. Accordingly, the probe unit 31 contains one or more electromagnetic radiation sources 32 and one or more detectors 33 which are located on the probe unit.

In an embodiment the probe unit is a transrectal probe. The transrectal probe will in use be located in the rectum of a subject and emitting electromagnetic radiation into tissue in its vicinity, up to approximately 10 cm radial.

In an embodiment the electromagnetic radiation source, such as a short-pulsed laser, is distantly located from the probe unit and connected to the probe unit via a electromagnetic radiation conductor, such as an optical fiber(s).

In an embodiment the probe unit is used to detect suspicious prostate tissue.

In an embodiment the probe unit further comprises an ultrasound source for emitting ultrasonic acoustic waves into the investigated tissue in order to image the geometry and location of the tissue. The detector unit comprising at least one ultrasound detector may in this embodiment be used to both detect ultrasound originating from the ultrasound source and ultrasonic acoustic waves originating from the photoacoustic effect due to the electromagnetic radiation from the electromagnetic radiation source. Whereas Photoacoustic Imaging in the near-infrared wavelength region is mainly sensitive to the water, lipid, Hb, and HbO2 content, use of the ultrasound source provides topographic details, such as the boundary of the prostate, the rectal wall, and the needle for a biopsy. An advantage of this embodiment is that the probe unit may be used to detect diseased tissue using the electromagnetic radiation source, ultrasound detector and image reconstruction unit, and then it may be used to guide a biopsy of the diseased tissue using the electromagnetic radiation source, the ultrasound source, ultrasound detector and the image reconstruction unit.

In an embodiment only one of the electromagnetic radiation source or the ultrasound source is active at every point in time. This means that image reconstruction unit will process received ultrasound information originating from the electromagnetic radiation source and the ultrasound source separately. This embodiment enables the image reconstruction unit to calculate separate image datasets for the two different sources. An advantage of this embodiment is that the same detector unit may be used to both detect ultrasound to detect different tissue types and to image the tissue using regular ultrasound imaging using the ultrasound source.

In a practical implementation, first the electromagnetic radiation source is active and the received ultrasonic information by means of the ultrasound detector will be processed by the image reconstruction unit to result in a first image dataset comprising the location of the diseased tissue based on the photoacoustic effect. Once the location of the diseased tissue is determined the electromagnetic radiation source will be deactivated and the ultrasound source will be activated. Using the ultrasound detector the received ultrasonic information, is then processed by the image reconstruction unit resulting in a second image dataset comprising the contours of the investigated tissue.

For image reconstruction the position between the electromagnetic radiation source and the detector unit with respect to each other has to be known. This is especially a problem if a combination of two probe units is used. The ultrasound unit may be used to determine the position and orientation of the probe unit or probes units with respect to each other.

If the ultrasound unit is incorporated into the transrectal probe the transurethral probe will be clearly visible.

In an embodiment the image reconstruction unit utilizes the first and the second image datasets to correlate their coordinate systems in order to create a third image dataset comprising information from both image datasets regarding location of diseased tissue from the first image dataset and the tissue contours of the second image dataset. While the ultrasound source is active, the image reconstruction unit will continuously create new second image datasets correlating the location of the diseased tissue to the new second image dataset creating new third datasets.

In an embodiment the combination of the using the ultrasound source and electromagnetic radiation source will improve the resulting image dataset from the image reconstruction unit, by overlaying both image datasets or by using anatomical information obtained by US for the image reconstruction of the optical image dataset.

In an embodiment the probe unit further comprises a biopsy unit that may be introduced into the tissue for taking a biopsy of a suspicious part of the tissue. The biopsy unit receives information from the image reconstruction unit regarding the exact location of the tissue type of interest, such as the diseased tissue. This embodiment has the advantage that the biopsy may be performed while continuously imaging the tissue. This eliminates problems with repositioning between a dedicated imaging and a dedicated biopsy tool. A user observing the continuously created third image datasets, which e.g. are presented on a display, may guide the biopsy needle, while the ultrasound source is activated. An advantage of this embodiment is that the number of false negatives will be drastically reduced, as the user knows the location of the diseased tissue.

In an embodiment the probe unit is a transurethral probe that may be inserted into the urethra and in use be located in the vicinity of the prostate gland. In use the electromagnetic radiation propagates through the prostate, so that the ultrasound signal from the back of the prostate, i.e. the urethral side, is stronger but has to travel further through the tissue.

In an embodiment the probe unit is an endoscope suitable for urethral, rectal or oral insertion and applications.

In use, in an embodiment, the probe unit is successively repositioned between each image reconstruction in order to image the prostate from several different angles. The created image datasets by the image reconstruction unit may be combined to give extended information of the imaged tissue. The image reconstruction unit may perform this combination using segmentation techniques commonly known in the field of image analysis.

In an embodiment the probe unit may be combined with a gel that enhances the optical contact between the probe unit and the surrounding tissue. The gel may be an ultrasound gel with scattering particles. In this embodiment the electromagnetic radiation source is located on the probe unit capable of emitting pulsed electromagnetic radiation.

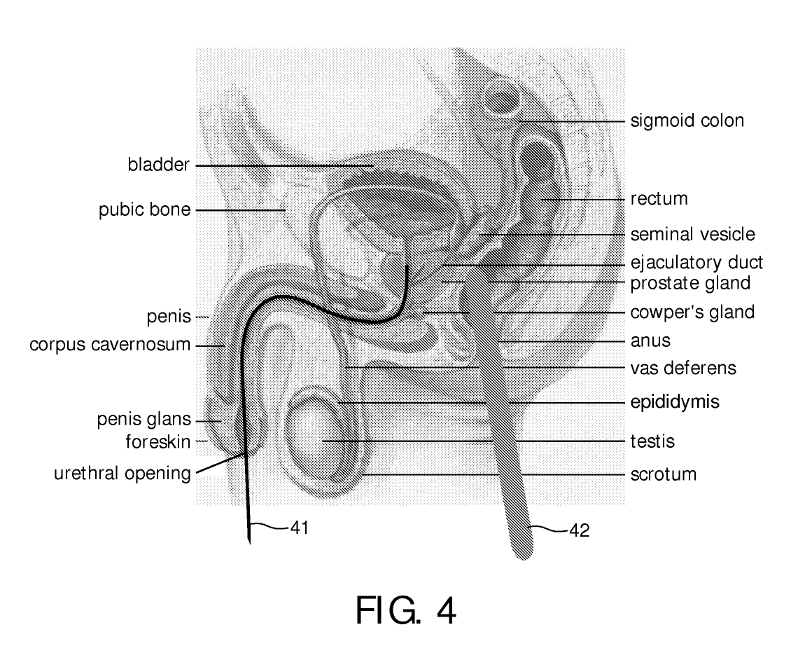

In an embodiment, according to FIG. 4, the system comprises two probe units; such as one transurethral probe 41 and one transrectal probe 42. The electromagnetic radiation source is located on the transurethral probe and is used to illuminate the prostate with pulsed electromagnetic radiation. The transrectal probe comprises an ultrasound detector for receiving generated ultrasonic acoustic waves corresponding to the photoacoustic effect by the emitted pulsed electromagnetic radiation from the transurethral probe. The transrectal probe is connected to the image reconstruction unit, which based on the received ultrasonic acoustic waves creates an image dataset of the investigated tissue as described above.

In an embodiment the transrectal probe comprises one or more of the electromagnetic radiation sources. In use the transurethral probe is placed in the urethra in the vicinity of the prostate. The transurethral probe comprises one or more detector units for receiving the ultrasonic acoustic waves generated by the photoacoustic effect by the electromagnetic radiation from the transrectal probe. In use the transrectal probe is placed in the rectum in the vicinity of the prostate.

In some embodiments the two probe units are positioned in such a way that the prostate is located between the probe units. More particularly, the probe units are positioned such that the emitted electromagnetic radiation from the transrectal probe propagates through the prostate and the detector unit(s) of the transurethral probe is positioned to receive the generated ultrasonic acoustic waves.

In an embodiment the probe unit is a bladder probe. The bladder probe has the shape of an umbrella that may be unfolded inside the bladder. The bladder may and contain electromagnetic radiation sources and/or detectors. In use the umbrella touches the bottom of the bladder to be as close a possible to the prostate region.

In another embodiment a saddle probe is comprised in the system. The saddle probe has the shape of a saddle and in use touches the genital area and contains electromagnetic radiation source(s) and/or detector(s).

In an embodiment a combination of transrectal, transurethral, and bladder probe is used for imaging of the prostate gland, wherein each probe unit may contain zero, one or more electromagnetic radiation sources, and zero, one or more detector units.

In an embodiment at least one of the probe units contains at least one electromagnetic radiation source and at least one of the probe units contains at least one detector unit.

In another embodiment the probe unit comprises an optical fiber, wherein the electromagnetic radiation source is located ex vivo.

The system according to some embodiments of the invention may be used for locating and diagnosing lesions in the human body in vivo. In some applications, once the exact position of a lesion is found a biopsy may be taken from the lesion using e.g. ultra sound techniques for guidance of the biopsy needle. Use of the system drastically reduces the negative biopsy samples compared to currently used “blind sampling” techniques. This reduces patient discomfort and minimizes infections as the number of biopsy samples is reduced. The biopsy may then be analyzed to determine the severity of the lesion. After the biopsy is analyzed a treatment of the lesion area may be performed to cure the patient. In other applications treatment may be performed without the need of a biopsy. Treatment of the diseased tissue may be performed using radiation therapy, chemotherapy etc.

In an embodiment the system may be used in combination with surgery for locating, diagnosing, and treating prostate cancer.

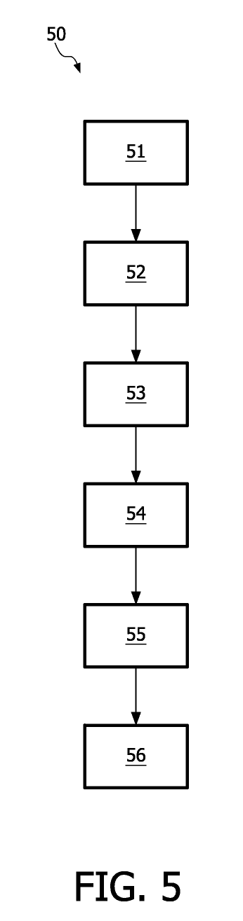

In an embodiment, according to FIG. 5, a method 50 for imaging of tissue in an anatomical structure is provided. The method comprises emitting 51 electromagnetic radiation into the anatomical structure from at least one electromagnetic radiation source, the electromagnetic radiation being absorbed in the anatomical structure exciting a first ultrasonic acoustic wave from the tissue due to thermo elastic expansion, receiving 52 the first ultrasonic acoustic wave by at least one detector unit, and reconstructing 53 a first image dataset of the tissue based on the received first ultrasonic acoustic wave.

In an embodiment the method further comprises emitting 54 a second ultrasonic acoustic wave into the anatomical structure, receiving 55 the second ultrasonic acoustic wave by the at least one detector unit, and reconstructing 56 a second image dataset of the tissue based on the received second ultrasonic acoustic wave.

In an embodiment a use of the method is provided to locate and diagnose a lesion in the human body in vivo.

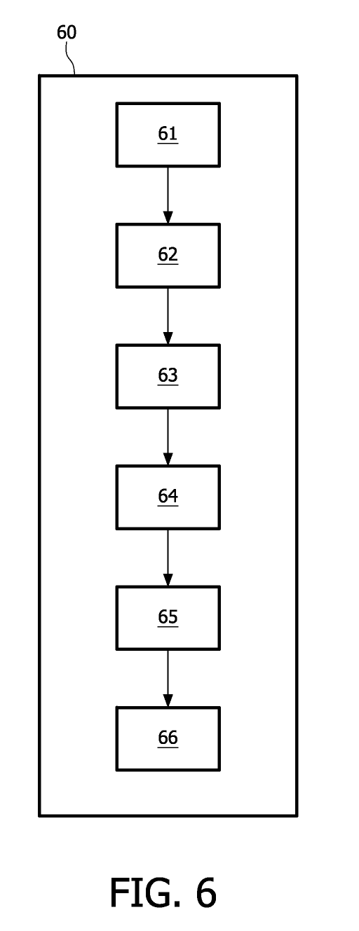

In an embodiment, according to FIG. 6, a computer-readable medium 60 is provided having embodied thereon a computer-program for processing by a computer for imaging of tissue in an anatomical structure. The computer program comprises a first emission code segment 61 for emitting electromagnetic radiation into the anatomical structure from at least one electromagnetic radiation source, the electromagnetic radiation being absorbed in the anatomical structure exciting a first ultrasonic acoustic wave from the tissue due to thermo elastic expansion, a first reception code segment 62 for receiving the first ultrasonic acoustic wave by at least one detector unit, and a first reconstruction code segment 63 for reconstructing a first image dataset of the tissue based on the received first ultrasonic acoustic wave.

In an embodiment the computer-readable medium further comprises a second emission code segment 64 for emitting a emitting a second ultrasonic acoustic wave into the anatomical structure, a second reception code segment 65 for receiving the second ultrasonic acoustic wave by the at least one detector unit, and a second reconstruction code segment 66 for reconstructing a second image dataset of the tissue based on the received second ultrasonic acoustic wave.

In an embodiment the computer-readable medium comprises code segments arranged, when run by an apparatus having computer-processing properties, for performing all of the method steps defined in some embodiments.

In an embodiment the computer-readable medium comprises code segments arranged, when run by an apparatus having computer-processing properties, for performing all of the functions of the system defined in some embodiments.

The invention may be implemented in any suitable form including hardware, software, firmware or any combination of these. However, preferably, the invention is implemented as computer software running on one or more data processors and/or digital signal processors. The elements and components of an embodiment of the invention may be physically, functionally and logically implemented in any suitable way. Indeed, the functionality may be implemented in a single unit, in a plurality of units or as part of other functional units. As such, the invention may be implemented in a single unit, or may be physically and functionally distributed between different units and processors.

Although the present invention has been described above with reference to specific embodiments, it is not intended to be limited to the specific form set forth herein. Rather, the invention is limited only by the accompanying claims.

In the claims, the term “comprises/comprising” does not exclude the presence of other elements or steps. Furthermore, although individually listed, a plurality of means, elements or method steps may be implemented by e.g. a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

1. A system (10) for in vivo imaging of tissue in an anatomical structure, said system comprising
a first unit (31, 41, 42) connected to at least one electromagnetic radiation source (11) for emitting pulsed electromagnetic radiation into said anatomical structure, whereby a first ultrasonic acoustic wave is generated from said tissue, said system further comprising
at least one ultrasound source (13) for emitting a second ultrasonic acoustic wave into said anatomical structure,
at least one detector unit (12) for receiving said first ultrasonic acoustic wave and said second ultrasonic acoustic wave,
an image reconstruction unit (14) for reconstructing a first image dataset of said tissue based on said received first ultrasonic acoustic wave and a second image dataset of said tissue based on said received second ultrasonic acoustic wave.

2. The system according to claim 1, wherein said image reconstruction unit is configured to calculate a third image dataset combining image dataset information from said first image dataset and said second image dataset.

3. The system according to claim 1, wherein said detector unit is located on said first unit.

4. The system according to claim 1, further comprising a second unit (31, 41, 42) having an electromagnetic radiation source for emitting pulsed electromagnetic radiation into said anatomical structure, whereby a third ultrasonic acoustic wave is generated from said tissue, wherein said detector unit is configured to receive said third ultrasonic acoustic wave, wherein said second unit is connected to said image reconstruction unit for reconstructing a fourth image dataset of said tissue based on said received third ultrasonic acoustic wave.

5. The system according to claim 4, wherein said detector unit is located on said second unit.

6. The system according to claim 1, wherein said ultrasound source is located on said first unit.

7. The system according to claim 1, wherein said first unit is a transrectal unit suitable for insertion via rectum or a transurethral unit suitable for insertion via urethra.

8. The system according to claim 4, wherein said second unit is a transurethral unit suitable for insertion via urethra or a transrectal unit suitable for insertion via rectum.

9. The system according to claim 7, wherein said transrectal unit and said transurethral unit in use are located in the vicinity of the prostate gland.

10. The system according to claim 1, wherein the image dataset reconstructed by the image reconstruction unit is a 2D, 3D, or multi-dimensional image dataset.

11. The system according to claim 1 wherein the distance between said at least one electromagnetic radiation source and said at least one detector unit is 2 mm to 10 cm.

12. The system according to claim 1, wherein said first image dataset is used to guide a biopsy of said tissue.

13. The system according to claim 1 being comprised in a medical workstation or medical system.

14. A method for imaging of tissue in an anatomical structure, said method comprising
emitting electromagnetic radiation into said anatomical structure from at least one electromagnetic radiation source, generating a first ultrasonic acoustic wave from said tissue,
receiving said first ultrasonic acoustic wave,
reconstructing a first image dataset of said tissue based on said received first ultrasonic acoustic wave,
emitting a second ultrasonic acoustic wave into said anatomical structure,
receiving said second ultrasonic acoustic wave, and
reconstructing a second image dataset of said tissue based on said received second ultrasonic acoustic wave.

15. A computer-readable medium (60) having embodied thereon a computer-program for processing by a computer for imaging of tissue in an anatomical structure, said computer program comprising
a first emission code segment (61) for emitting electromagnetic radiation into said anatomical structure from at least one electromagnetic radiation source, whereby a first ultrasonic acoustic wave is generated from said tissue,
a first reception code segment (62) for receiving said first ultrasonic acoustic wave,
a first reconstruction code segment (63) for reconstructing a first image dataset of said tissue based on said received first ultrasonic acoustic wave
a second emission code segment (64) for emitting a second ultrasonic acoustic wave into said anatomical structure,
a second reception code segment (65) for receiving said second ultrasonic acoustic wave,
a second reconstruction code segment (66) for reconstructing a second image dataset of said tissue based on said received second ultrasonic acoustic wave.

16. Use of the system according to claim 1 for locating and diagnosing a lesion in a tissue in an anatomical structure in vivo.

17. Use of the system according to claim 1 for guiding a biopsy of a lesion in a tissue in an anatomical structure in vivo.
